(19) United States
(12) Patent Application Publication
(10) Pub. No.: US 2007/0014728 A1
(43) Pub. Date: Jan. 18, 2007

(54) Device and method for increasing the throughput of irritation testing of transdermal formulations

(76) Inventors: Delphine C. Imbert, Cupertino, CA (US); Paul A. Johnson, Los Banos, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/489,428

(22) Filed: Jul. 18, 2006

Related U.S. Application Data
(60) Provisional application No. 60/700,076, filed on Jul. 18, 2005.

(51) Int. Cl. A61B 10/00; A61K 49/00

(52) U.S. Cl. 424/9.8

(57) ABSTRACT

A flexible test device and a method for irritation testing of formulations on skin. The test device includes a flexible substrate, a group of formulations secured on the flexible patch for application on the skin of an animal such that the test device conforms to the skin for the formulations to contact the skin, and adhesive for attaching the test device to the skin for a predetermined period of time.

### CROSS REFERENCE TO RELATED U.S. APPLICATION INFORMATION

The present application claims the benefit under 35 U.S.C. 119(e) of U.S. Provisional application 60700,076 filed Jul. 18, 2005.

### FIELD OF THE INVENTION

This invention relates to methods and devices for testing irritation of compositions on skin. In particular, the invention relates to methods and devices for testing a large number of compositions suitable for transdermal drug delivery.

### BACKGROUND

For a long time people have been applying ointments, creams, lotions, gels, and liquids containing drugs on skin surfaces to treat skin ailments. The skin, however, is a natural barrier to many drugs and presents a challenge to drug delivery into the systemic circulation. More recently, people have invented devices to provide transdermal delivery of drugs, even to the systemic circulation. Transdermal drug delivery can generally be considered to involve two groups: transport by a “passive” mechanism or by an “active” transport mechanisms. In the former, such as the fentanyl transdermal systems made by ALZA Corporation, the drug is incorporated in a solid matrix, a reservoir, and/or an adhesive system. In the “active” type of transdermal drug delivery the flux of the drug(s) is driven by various forms of energy. Some examples include the use of iontophoresis, ultrasound, electroporation, heat, and microneedles.

Whether for passive or active transdermal drug delivery, to facilitate delivery, permeation enhancers and/or other excipients are often included with the drug in a composition that is applied on the body surface of an individual, e.g., a patient for drug delivery. However, not all drugs, excipients, and compositions containing a drug/excipient combination are suitable for delivery on the skin. Many factors contribute to whether a drug or drug composition is suitable for dermal application, e.g., as in transdermal delivery. These include irritation, delivery characteristics (as measured by in vitro skin flux or clinical studies), adhesive properties/wear, and formulation stability.

Irritation is the local inflammatory response of normal living skin to direct injury by single, repeated, or prolonged contact with a chemical agent without the involvement of an immunologic mechanism. The irritation properties of a drug often determine whether it can be delivered via the skin. Erythema and edema are macroscopic manifestations of irritation. Generally, the testing procedures for determining primary skin irritation used by scientists in the transdermal field are based on the methods described by Draize, Woodward, and Calvery (Prevo M, Cormier M, Nichols K. Predictive Toxicology Methods for Transdermal Delivery Systems. Toxicology Methods 1996; 6(2): 83-98) and adopted in the CFR, 1500.41 in the Federal Hazardous Substances Act, Chapter II, Title 16. Typically such testing is conducted in rabbits, but guinea pigs have been found to be a useful model as well (Chester A E, Terrell T G, Nave E, Dorr A E, DePass L R. Dermal sensitization study in hairless guinea pigs with dinitrochlorobenzene and ethyl aminobenzoate. J Toxicol Cutan Ocul Toxicol 1988; 7(4): 273-281). Both hairless guinea pigs and rabbits may be used to comply with government regulations, which request evaluation in two species.

In irritation studies, a maximum of 4-6 formulations is typically tested per animal and about 1.5-2.5 cm² of skin are exposed to each formulation. Prior to the application of a transdermal system or its components, skin sites are wiped with an isopropyl alcohol swab. Sites are allowed to air dry or be blotted dry with gauze. If test articles are liquid (e.g., diluted or undiluted components, such as:permeation enhancers) 0.1 mL is placed in a Finn chamber or 0.4 mL in a Hilltop chamber, with or without gauze, and applied to the skin site using MICROPORE® tape. After a maximum of 7 days, the test articles are removed. Duration of application depends on clinical indication and/or research objectives. All treatment sites are scored for erythema, eschar formation, and edema at approximately 0.5, 24 and 48 hours after removal of the test article. The method of scoring of erythema and edema is well known by people skilled in the art. Formulations are usually tested in 6 replicates. Thus, for 36 test formulations, 216 testing systems are needed to be tested on as many as 54 animals. Testing such a large number of systems on so many animals requires a significant amount of resources and time. Therefore, better devices and methods of testing formulations (for chemicals, drugs, enhancers, excipients, etc.) on live animals are needed.

### SUMMARY

The present invention provides devices and methods for testing formulation compositions for irritation to the skin of animals. In one aspect, a method for irritation testing involves applying a test device (e.g., a patch) with a flexible substrate having a group of formulations on the skin of an animal. The test device conforms to the skin such that the formulations can contact the skin and remain in position for a predetermined period of time. In an aspect of the invention, the formulations can be arranged in an array on the substrate. In another aspect, an adhesive is provided in the test device to attach the test device on the skin.

In another aspect, a method of making a test device for testing irritation on skin is provided. The method includes securing a group of formulations on a flexible substrate to form a test device, such that the test device conforms to the skin for the formulations to contact the skin, and attaching the test device to the skin for a predetermined period of time. In an aspect of the invention, the formulations can be arranged in an array on the substrate. In another aspect, an adhesive is provided in the test device to attach the test device on the skin.

In yet another aspect, a flexible test device is provided for irritation testing of formulations on skin. The test device includes a flexible substrate, a group of formulations secured on the flexible patch for application on the skin of an animal such that the test device conforms to the skin for the formulations to contact the skin, and adhesive for attaching the test device to the skin for a predetermined period of time. In an aspect of the invention, the formulations can be arranged in an array on the substrate. In another aspect, an adhesive is provided on the substrate facing the skin to attach the test device on the skin.

The techniques and devices of the present invention allow for irritation testing of a large number of formulations on a relatively small number of animals at a relatively fast pace. Test formulations are positioned on a flexible, conformable substrate in group(s) format, thus allowing a plurality of test formulations to have simultaneous contact with the skin surface.

Since the test formulations are arranged in an array format and each array holds in the order of many, such as about 9 or more (e.g., about 9-50) test formulations and multiple arrays can be positioned on each animal, many formulations can be tested simultaneously. The manufacture of the array can be conducted manually or by an automated dispensing robot, which results in significant reduction of manufacture time and potential formulation errors. In addition, time needed for handling animals is also significantly reduced because test formulations can be applied, e.g., 9-50 at a time on an animal instead of one by one. Also, because fewer animals are required, the time involved in moving them in and out of the cages, as well as applying and removing protective bandages is also reduced. Overall, with the present invention, the procedure of irritation testing is simplified, execution time is reduced, throughput is increased, and fewer animals are needed. These advantages allow such testing to be incorporated much earlier in the formulation development process. Furthermore, in the case where humans are the subjects, using the present invention greatly reduces the inconvenience and discomfort to both the physician and the patient.

### BRIEF DESCRIPTION OF THE DRAWINGS

The present invention is illustrated by way of example in embodiments and not limitation in the figures of the accompanying drawings in which like references indicate similar elements. The drawings are not drawn to scale unless indicated otherwise.

FIG. 1 is a schematic illustration of a plan view of an embodiment of the test device of the present invention.

FIG. 2 is a schematic illustration of a sectional view of an embodiment of the test device of the present invention.

FIG. 3 is schematic illustration of a sectional view of another embodiment of the test device of the present invention, showing raised surfaces.

FIG. 4 is schematic illustration of a sectional view of another embodiment of the test device of the present invention, showing formulations with portions set in depressions.

FIG. 5 is schematic illustration of a sectional view of another embodiment of the test device of the present invention, showing a lid.

FIG. 6 is schematic illustration of a sectional view of yet another embodiment of the test device of the present invention.

FIG. 7 is schematic illustration of a sectional view of yet another embodiment of the test device of the present invention.

FIG. 8 is an illustration of a test device of the present invention applied to a guinea pig.

FIG. 9 is a graph showing the comparison of irritation scores between array test and conventional test. C, D, E and F represent various transdermal matrix formulations.

FIG. 10 is a graph showing the comparison of irritation scores on guinea pigs of a test done with a device of the present invention with irritation scores of a test done with conventional protocol.

FIG. 11 is a graph showing the comparison of irritation scores on rabbits of a test done with a device of the present invention with irritation scores of a test done with conventional protocol.

FIG. 12 is a graph showing the comparison of irritation scores on guinea pigs and rabbits of a test done with a device of the present invention with irritation scores of a test done conventionally, combining the data of FIG. 10 and FIG. 11.

### DETAILED DESCRIPTION

In describing the present invention, the following terms are intended to be defined as indicated below. As used in this specification and the appended claims, the singular forms “a,” “an” and “the” include plural references unless the content clearly dictates otherwise.

As used herein, the term “transdermal” refers to the use of skin, mucosa, and/or other body surfaces as a portal for the administration of drugs by topical application of the drug thereto for passage into the systemic circulation.

“Pharmaceutical agent” is to be construed in its broadest sense to mean any material that is intended to produce some biological, beneficial, therapeutic, diagnostic or other intended effect, such as relief of pain and contraception. Unless specified differently in context, as used herein, “drug” and “pharmaceutical agent” are used interchangeably herein.

As used herein, the term “permeation enhancer” refers to chemical agents that increase the permeability of skin to a drug in the presence of the chemical agent as compared to permeability of skin to the drug in the absence of the chemical agent.

The term “intact body surface” refers to a body surface, such as skin, that does not have wounds or crevices, and has not been punctured by sharp objects.

The present invention provides devices and methods for high throughput testing of compositions for irritation on skin of animals. The present technique utilized a flexible device having a group of formulations (i.e., different compositions containing chemicals) to be tested. The device can be applied as a patch on to the skin surface of an animal (e.g., rabbit, guinea pig, dog, pig, human) for a period of time for evaluation of irritation symptoms afterward.

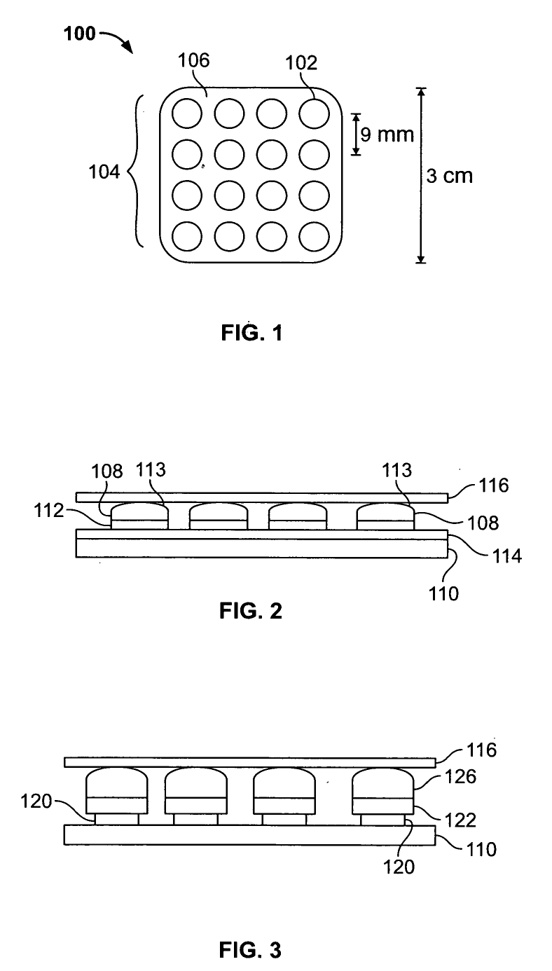

In an embodiment shown in FIG. 1, the invention includes a test device 100 having a large number of test formulation dots 102 grouped in an array 104. The array is about 3 cm×3 cm (for testing in guinea pigs) and is affixed on a substrate (backing material) 106. Each formulation dot has a generally circular disk shape and is about 8 mm in diameter. The size of the each formulation dot and its distance from an adjacent neighbor are chosen such that skin reactions can be readily identified. Test formulations are dispensed as disk-shaped dots 102 (FIG. 1) or other alternative shapes on the array substrate 106. The disk shape is a result of ease for forming, e.g., by cutting or drop deposition. Other alternative shapes such as oval, polygonal (e.g., hexagonal, square), etc., can also be used. Generally, a formulation of at least about 12 mm is useful, preferably about 15 mm² to 200 mm², more preferably about 25 mm² to 100 mm². Dot diameters (or the largest lateral dimension) can range from about 2mm to 15 mm, preferably 3 mm to 12 mm. A test formulation is spaced apart from a neighbor formulation with a gap of, preferably, about 2 mm to 5 mm. The center of each formulation dot can be about 6mm to 20 mm, preferably 10 mm to 16 mm, from the center of a neighboring formulation dot. Preferably, a test device for a hairless guinea pig has at least 9 test formulations, preferably at least 15 formulations, more preferably about 9 to 50 test formulations arranged as a single group. The test formulations can be evenly spaced in the single group. Multiple groups can be present in a larger test device if adhesive is provided to maintain contact with skin for all of the groups. Although convenient to arrange, a row by column array is only one of many choices for an array, which is a regular, ordered pattern of grouping for ease of identification of the individual test formulations in the arrangement. Other possible arrangements include dotted spiral, dotted concentric rings, dotted honey comb pattern, etc.

The optimal area size and separation for the test formulation dots are selected such that they are large enough for scoring erythema and edema but small enough to prevent crosstalk (i.e., interference of signs of erythema and edema between adjacent neighbors). Such sizing and separation can be determined experimentally. If species other than hairless guinea pigs are considered for irritation testing, size can be adapted to the anatomy of the animal. For example, larger arrays could be used in rabbits (about 10 cm×8 cm having about 63 formulation dots). The size of the device is selected so that the device can be pressed to conform and adhere to the skin surface in order-for all formulation dots to adequately contact the skin surface. Alternatively, more than 2 arrays could be placed on larger animals. Two 3 cm×3 cm array fit nicely (with good skin contact and without ridges) on the flanks of a hairless guinea pig.

The substrate 106 used for backing support for the array should be flexible enough so that it conforms to the body site where it is applied and preferably stays in contact with the intact skin of the animal at all times i.e., continuously during the test period. The substrate may be formed from a flexible material such as a breathable or occlusive material, which may be a fabric or sheet made of polyvinyl acetate, polyvinylidene chloride, polyethylene, polyurethane, polyester, ethylene vinyl acetate (EVA), polyethylene terephthalate, polybutylene terephthalate, coated paper products, aluminum sheet, and the like, or a combination thereof. The substrate may be low density polyethylene (LDPE) materials, medium density polyethylene (MDPE) materials or high density polyethylene (HDPE) materials, e.g. SARANEX (Dow Chemical, Midland, Mich.). The substrate may be a monolithic or a multilaminate layer. In certain embodiments, the substrate is a multilaminate layer including nonlinear LDPE layer/linear LDPE layer/nonlinear LDPE layer. The substrate can have a thickness of about 0.012 mm (0.5 mil) to 0.125 mm (5 mil); preferably about 0.025 mm (1 mil) to 0.1 mm (4 mil); more preferably about 0.0625 mm (1.5 mil) to 0.0875 mm (3.5 mil). The substrate can also be made by weaving, knitting, or fibers glued together. Other examples of substrates available commercially is MEDPAR® multilaminate (which is a medium density polyethylene/aluminum foil/PET/EVA laminate), spun-bound polyester or other non-woven backing like POROUSEAL, KENDALL, etc.

Test formulations can include any transdermal dosage forms, including patches, ointments, gels, creams, and lotions. It is understood that the term “patches” when referred to formulation dots (as in “dot patches”) refers to the small formulation dots (in a patch form and shape) in the array that is in turn on the testing device 100, which itself can be in a patch form (although larger than the smaller formulation patch dots). Such formulation dot patches may be disk shaped (or other alternative shapes). Such formulation dot patches, for example, can be cut as a small piece from a traditional transdermal drug delivery patch, or made separately. The present invention is well suited for testing transdermal patches of the matrix type, i.e., where drug and excipients are dissolved in a pressure-sensitive adhesive in contact with the skin.

When the test formulations are matrix patches, arrays can be prepared using an automated dispensing robot. Robotics, including programmable equipment that can dispense materials as films, dry, form laminate, cut, arrange, form arrays, and the like, are known in the art and will not be described in detail herein. Instead of using automated means, test devices with arrays can also be prepared manually.

As shown in FIG. 2, special features can be implemented in the design of the substrate 106 to prevent the test formulation dots from touching one another, particularly during dispensing. In FIG. 2, the test formulations 108 are affixed and spaced from the substrate 110 with a spacer layer 112, which itself can be an adhesive. The test surfaces 113 of the formulations 108 are raised to create more spacing from the adhesive layer 114. In this way, when the dots are dispensed as a liquid (e.g., ethyl acetate solution) before drying into an adhesive dot, spreading to a neighbor dot is prevented. Also, when the device is applied by pressing on the skin surface, even if the test formulations 108 is slightly deformed and spread a little it will not reach or be too close to a neighbor formulation. Further, the adhesive layer 114 is far enough from the surface 113 of the test formulation 108 that, when pressed on the skin, the adhesive will not touch the skin, thereby eliminating any effect on the skin, such as irritation, that may be caused by the adhesive. The thickness of the spacer layer can be chosen such that the surface 113, is at a gap space of about 0.5 mil (0.0125 mm) to 25 mil (0.625 mm), preferably about 1 mil (0.025 mm) to 5 mil (0.125 mm) from the surface from which the test formulation projects, be it an adhesive surface, a substrate surface, or some other surface. This spacing can vary as a function of the size of the test formulation. If the test formulation 108 has the tendency to spread when pressed firmly, the test surface 113 of the test formulation can be made slightly curved convexly such that when pressed on the skin, the test formulation will conform to the skin surface without spreading to an area near to a neighboring test formulation. In one aspect, the curved surface need not be symmetrical so long as the middle part gradually rises from the edge. A release liner 116 can be used to cover and protect the array of formulations 108.

As shown in FIG. 3, in the case of the raised-surface design, alternatively, the adhesive 120 can be located on the substrate 110 entirely under the spacer layer 122, such that none of the adhesive is exposed outside of the spacer layer 122 viewing perpendicularly from the plane of the substrate 110. This would further ensure that even when the device 124 is pressed firmly on the skin, the adhesive 120 does not contact the skin. The thickness of the spacer layer 122 can be chosen while considering the thickness of the adhesive layer 120. If the adhesive 120 is just a thin film, its thickness may be negligible. If the adhesive 120 has a thickness that may have effect on the gap from the release liner 116 to the substrate 110, the thickness of the spacer layer 122 will be chosen accordingly. A design with a spacer layer is especially useful for cases where the adhesive might cause irritation to confound irritation scores of test formulation. The spacer layer 112, 122 can be manufactured from low-nickel content metal, polymeric material, or any other material that prevents the diffusion of components of the test formulations into the adhesive layer but does not irritate or sensitize the skin. A release liner 116 covers and protects the array of formulations 126.

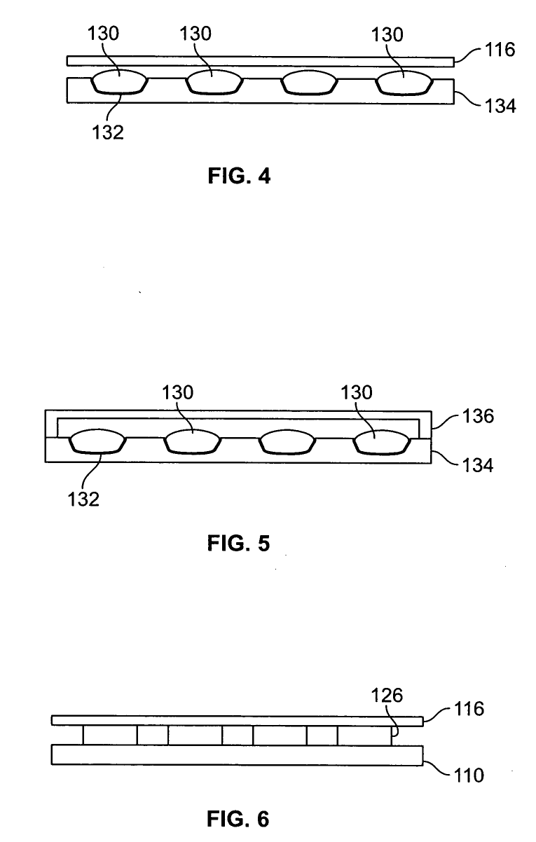

Instead of using a layer of adhesive to affix the test formulations on the substrate, an alternative way to hold the test formulations, as shown in FIG. 4, is to lay the test formulations 130 in depressions (or cavities) 132 in the substrate 134. The depression can have various sectional shapes so that the test formulation can be held without using an adhesive. For example, the bottom of the depression (or cavity) can be slightly bigger than the top so that once a portion of the test formulation is laid or pushed therein it will not fall out. If desired, an adhesive can be used in the depression to secure the test formulation to the depression. With a depression, the adhesive, if used, could be laid such that it will not be exposed to contact the skin. Again, a release liner 116 covers and protects the array of formulations 130.

FIG. 5 shows an embodiment in which a lid 136, instead of a release liner, is used to protect the test formulations 130. Although FIG. 5 shows the formulations being set in depressions 132 in portion, other designs, with or without depressions, spacers, etc., can still have a lid used for protecting the test formulations.

FIG. 6 shows yet another embodiment in which the test formulations 126 are set directly on the formable substrate 110 without a spacer layer. In such cases, the test formulation would need to have adequate adhesive property to stay on substrate 110, or a thin layer of adhesive can be used to affix the test formulation 126 to the substrate 110. For testing of semi-solid transdermal formulations (e.g., ointments, gels, creams, lotions), a design similar to that shown on FIG. 4 can be used, except the release liner can be replaced with a lid as shown on FIG. 5.

In certain embodiments, the test formulations have adequate adhesive property that they can stay on the skin during the test period without additional adhesive aid. In other cases, an adhesive is provided in the test device to affix the test device on the skin. FIG. 7 shows an embodiment of a test device 140 in which the array is a pattern of groups 142 of test formulations 144 affixed on the substrate 146. Adhesive 148 suitable for adhering to the skin is located on the substrate around the groups of the test formulations 144 so that the test device can adhere to the skin. Adhesive is also provided between the groups such that a large skin area can be covered without any test formulation contacting the skin inadequately. The adhesive can be located to encircle individual groups of test formulation (covering the area between the groups) or encircle all the groups together but not covering the area between the groups. If desired, adhesive tapes can be used to span over the device to attach to the skin. Any adhesive tape compatible with skin can be used, including commercially available first aid tapes or patches. In this case, the substrate of the test device may or may not be made with a skin adhesive.

The test formulations can be made with various drugs (or agent to be tested) and matrix or reservoir material for containing the drugs or agents. Pharmaceutical agents and chemical entities that can be tested with the present invention include any chemical that is a candidate for skin irritation testing, such as therapeutic agents, excipients used in pharmaceutical dosage forms, diagnostic agents, suspected allergens, potential pharmaceutical products. These can be organic or inorganic materials, including analgesics, hormones, nervous system acting materials, steroids, agents that act on blood (e.g., anticoagulants), antiepileptic agents, antimicrobial agents, vasodilators, vitamins, transdermal permeation enhancers and other transdermal formulation excipients, proteins, polypeptides, polynucleotides, genes, RNA's, DNA's, and the like, alone or in combination. It is further noted that electrolytes, or other ingredients that can be held or dissolved in a composition that can be incorporated into a test formulation (e.g., in a matrix such as a gel) can be tested.

The test formulation can be formed with a carrier for holding the drug or test agent to be tested. The carrier is typically a matrix in which the drug or agent is dissolved or absorbed and from which the drug and agent can diffuse and permeate into the skin. Matrix material for forming formulations that can be applied to skin surfaces are described by, e.g., U.S. Pat. No. 6,181,963 and US Patent Publication 20040213832, which are incorporated by reference herein in their entireties. For example, the matrix can be formed from a polymeric material in which the drug or the excipient has reasonable solubility for the drug to be delivered within the desired range, such as, a polyurethane, ethylenevinyl acetate copolymer (EVA), polyacrylate, styrenic block copolymer, and the like. In certain embodiments, the matrix is formed from a pharmaceutically acceptable pressure sensitive adhesive, preferably a polyacrylate or a styrenic block copolymer-based adhesive. Examples of styrenic block copolymer-based adhesives include, but are not limited to, styrene-isoprene-styrene block copolymer (SIS), styrene-butadiene-styrene copolymer (SBS), styrene-ethylenebutene-styrene copolymers (SEBS), and di-block analogs thereof. Adhesives that can be used for forming the matrix include polyisobutylene and silicone based adhesives such as polydimethylsiloxane.

Acrylic polymers suitable for forming test formulations include copolymers or terpolymers with two or more exemplary components selected from the group including acrylic acids, alkyl acrylates, methacrylates, copolymerizable secondary monomers or monomers with functional groups. Examples of monomers include, but are not limited to, acrylic acid, methacrylic acid, methoxyethyl acrylate, ethyl acrylate, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, hydroxyethyl acrylate, hydroxypropyl acrylate, acrylamide, dimethylacrylamide, acrylonitrile, dimethylaminoethyl acrylate, dimethylaminoethyl methacrylate, tert-butylaminoethyl acrylate, tert-butylaminoethyl methacrylate, methoxyethyl acrylate, methoxyethyl methacrylate, vinyl acetate, and the like. Additional examples of appropriate acrylic adhesives suitable in the practice of the invention are described in Satas, “Acrylic Adhesives,” Handbook of pressure-Sensitive Adhesive Technology, 2nd ed., pp. 396-456 (D. Satas, ed.), Van Nostrand Reinhold, New York (1989). Acrylic adhesives are commercially available (National Starch and Chemical Corporation, Bridgewater, N.J.; Solutia, Mass.).

Furthermore, the reservoir material or matrix can be a gel in which a liquid, e.g., aqueous solution can be held. The gel can be formed of a hydrophilic polymer that is insoluble or soluble in water. Such polymers can be blended with the components in any ratio, but preferably represent from a few percent up to about 50 percent by weight of the reservoir. The polymers can be linear or crosslinked. Suitable hydrophilic polymers include copolyesters such as HYTREL™ (DuPont De Nemours & Co., Wilmington, Del.), polyvinylpyrrolidones, polyvinyl alcohol, polyethylene oxides such as POLYOX (Union Carbide Corp.), CARBOPOL™. (BF Goodrich of Akron, Ohio), blends of polyoxyethylene or polyethylene glycols with polyacrylic acid such as POLYOX.R™, blended with CARBOPOL™, polyacrylamide, KLUCEL™, cross-linked dextran such as SEPHADEX™ (Pharmacia Fine Chemicals, AB, Uppsala, Sweden), WATER LOCK™ (Grain Processing Corp., Muscatine, Iowa) which is a starch-graft-poly(sodium acrylate-co-acrylamide) polymer, cellulose derivatives such as hydroxyethyl cellulose, hydroxypropylmethylcellulose, low-substituted hydroxypropylcellulose, and cross-linked Na-carboxymethylcellulose such as Ac-DiSol (FMC Corp., Philadelphia, Pa.), hydrogels such as polyhydroxyethyl methacrylate (National Patent Development Corp.), natural gums, chitosan, pectin, starch, guar gum, locust bean gum, and the like, along with blends thereof. Of these, polyvinyl alcohols are preferred in an amount ranging from about 5 to about 35% by weight, preferably from about 19 to about 23% by weight of the contents of the reservoir. This list is merely exemplary of the materials suited for use in this invention. Other suitable hydrophilic polymers can be found in J. R. Scott & W. J. Roff, Handbook of Common Polymers (CRC Press, 1971), which is hereby incorporated by reference.

The test devices can be made by forming the test formulations of the appropriate size, providing the substrate and affixing the test formulations on the substrates with or without adhesive and covering with a lid or release liner. Commonly known release liner materials, adhesive materials, and substrate materials can be made or obtained commercially by persons skilled in the art. General knowledge of making transdermal formulations is also within the skill of a person skilled in the art. A larger cast of test formulation can be made first and then cut to the appropriate size for putting into an array on a substrate. Certain formulations can be dispensed in liquid form as dots directly on a substrate and allowed to solidify, e.g., by evaporation of solvent.

Knowing what drug(s) (or agent(s)) to test, and the matrix suitable for such drugs, test formulations can be made. With the teaching of the present application on the design of arrays and the technique of spacing and securing the formulations to the substrate, one skilled in the art will be able to make the test device with an array of test formulation for irritation testing on a test animal.

To test formulations, the test device is placed over an area of relatively area of the skin of a test animal and gently pressed thereon to affix the device on the skin such that the test formulations are in full contact with the skin. The substrate of the test device is adequately soft so that the device can conform to any undulation on the skin. This way, all of the test formulations would be in contact with the skin of the chosen site. The skin area suitable for such testing is, for example, the dorsal flank of a four legged mammal, e.g., dog, guinea pig, rabbit, rat, etc. For testing on a primate, e.g., human, the arm, thigh, back, abdomen, chest, or back area can be used. Prior to any application, hair should be clipped to provide a smooth surface and the area wiped with an alcohol swab to clean any excess oils or dander. Alternatively, hairless animals can be used. The skin should be prepared in the same manner. The hairless animals offer a time-effective alternative to haired animals as depilation or clipping hair is not required. In addition, undue irritation is reduced during removal of bandages, and application sites are not obscured by hair growth during scoring. The test device can be retained on the body surface for a period of about 1 day to 7 days, preferably continuously. The adhesive on the device should be adequately strong to maintain the device on the body surface for the desired period of time.

After the test device has been placed on the skin surface for the pre-determined duration and subsequently removed, the skin can be scored for skin irritation. Treated area is placed under a Luxo Color Correct Portable Lamp, or equivalent, to provide balanced, simulated daylight for all evaluations of skin treatment sites. All treatment sites are scored for erythema, eschar formation, and edema at approximately 0.5, 24 and 48 hours after removal of the test article.

In the analysis on irritation by inspection for edema and erythema, a score that can range from a minimum of 0 to a maximum of 4 is given for both edema and erythema. The edema and erythema scores are then added to arrive at an overall Primary Irritation Index (PII) score. The maximum PII is 8 and formulations may be categorized according to the following categories in Table I.

TABLE I

| PII | Category |
|---|---|
| 0.0-0.5 | None to negligible |
| 0.6-2.0 | Mild |
| 2.1-5.0 | Moderate |
| 5.1-8.0 | Severe |

### EXAMPLES

The following examples illustrate how the arrays can be used for testing formulations for irritation. They are for illustrative purposes only and one skilled in the art will know how to modify the techniques and designs to adapt to the chemical agents, matrix, and animal to be tested.

### Example 1

In formulation development and screening, exposure of 24 hours is typical. Later in development, exposure of 3 to 7 days may be used. Arrays are then removed and each sites is scored for edema and erythema at 0.5 hr, 24 hr, and 48 hr following removal of the arrays and using the same scales (0-4) as in the conventional primary skin irritation tests. A primary irritation index (PII) for each test formulation is calculated by adding the erythema score and the edema score at the 0.5-hr and 48-hr time points for each site where the formulation appeared, and dividing by the number of observations.

Arrays of formulations were applied on the flanks of the test animal, secured with MICROPORE® tape and/or bandages and left in place for 24 hours. A device with structures similar to that shown in FIG. 6 was used.

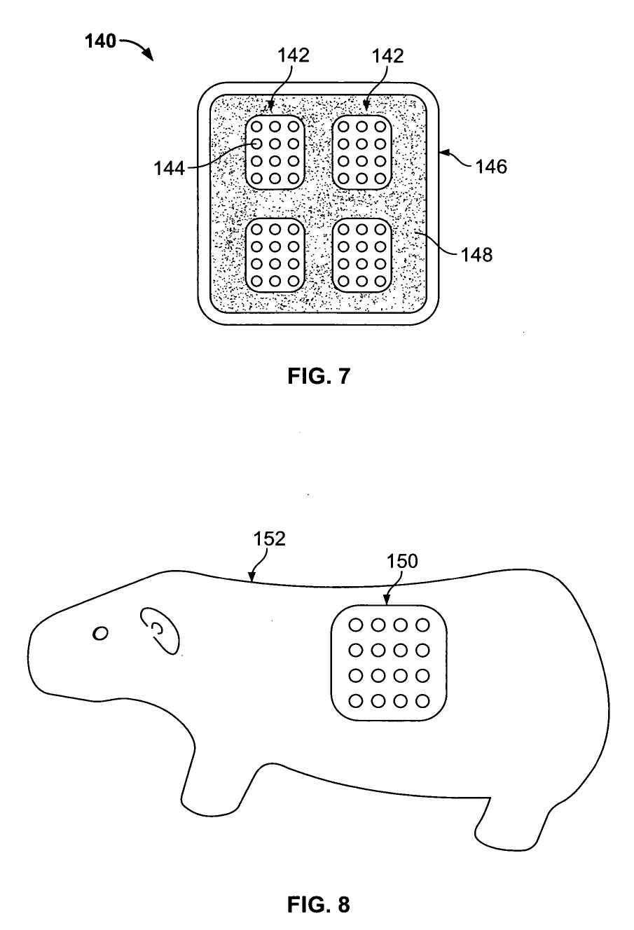

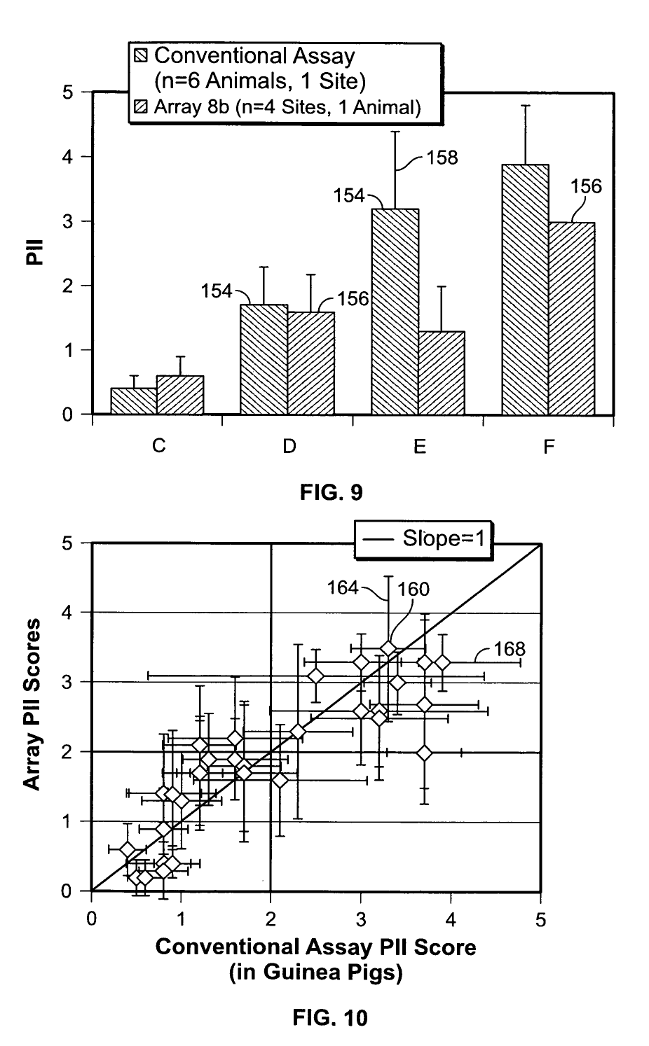

FIG. 9 shows the results of an irritation study comparing the conventional assay with results obtained from a prototype array for 4 different transdermal formulations containing drug and permeation enhancers in a matrix-type patch and spanning a wide range of PIIs. The prototype array was of a design as shown on FIG. 6 with a backing made of MEDPAR® multilaminate and 16 dots of a pressure-sensitive adhesive containing one drug and various enhancers. The formulations were picked to span a range of PIIs that is commonly encountered to result in test scores of 0-4. The scores for edema and erythema were read using the aforementioned method. The array used in this study was 4.8 cm² in overall area and contained 4 rows and 4 columns of evenly spaced test formulation dots each having an area of 30 mm² and separated from the neighbors with a gap of 4.5 mm. The arrayed test device 150 was placed on the flank of a hairless guinea pig 152 as shown in FIG. 8. The test formulation compositions were designated as C, D, E, and F, and were the same for both groups. For each formulation, C, D, E, or F, the bar 154 on the left indicates the averaged (i.e., mean) PII score for the conventional test and the bar 156 on the right indicates averaged PII score for the array test. For each bar a line extends from the top to indicate standard deviation of the measurement. The result of the tests done with the array indicated that there was no crosstalk between neighboring formulations (in that if there was any sign of irritation on a dot formulation on the array, it did not reach far enough to affect accurate scoring of the neighboring dot formulation). The PII scores of the tests done with the prototype array were reasonably close to those obtained by the conventional test.

### Example 2

In this study, 32 formulations containing a synthetic hormone and various enhancers in a pressure sensitive polyacrylate adhesive were tested using an improved design of the array. The arrays were constructed in a similar way as the device of FIG. 3. Each array had 16 formulation dots (6 mm in diameter) on 5 mil (0.127 mm), 401 stainless steel, chemically etched support positioned on a 1 mil (i.e., 0.025 mm) polyimide substrate with 9 mm spacing between each formulations. Two arrays holding 16 formulations each (4 rows×4 columns) were positioned on each flank of a hairless guinea pig and secured with tape and bandages. The 32 formulations were randomized across the 2 arrays on each animal. A total of 6 animals were used. Both hairless guinea pigs and rabbits were evaluated.

The formulations tested in this study were tested in an earlier study using the conventional PII protocol in hairless guinea pigs (IAF:HA-HO-hr, RCC Ltd Switzerland). Results from both the conventional and the array protocol are shown in Table II for both animal species and displayed on FIGS. 10-12.

TABLE II
PII Scores from Conventional Protocol vs. Array protocol

| | Conventional | | | | | | | | |
|---|---|---|---|---|---|---|---|---|---|
| | test | | | Array | | | | | |
| | (Hairless | | | (Hairless | | | Array | | |
| | Guinea Pigs) | | | Guinea Pigs) | | | (Rabbits) | | |
| Formulation | Mean | sd | n | Mean | Sd | n | Mean | sd | n |
| #1 | 0.5 | 0.0 | 6 | 0.2 | 0.26 | 6 | 1.5 | 0.8 | 6 |
| #2 | 1 | 0.4 | 6 | 1.3 | 0.69 | 6 | 0.6 | 0.5 | 6 |
| #3 | 2.1 | 1.0 | 6 | 1.6 | 0.80 | 6 | 1.8 | 0.8 | 6 |
| #4 | 0.6 | 0.2 | 6 | 0.2 | 0.26 | 6 | 1.6 | 0.2 | 6 |
| #5 | 0.8 | 0.3 | 6 | 0.9 | 0.58 | 6 | 1.1 | 0.5 | 6 |
| #6 | 1.2 | 0.4 | 6 | 1.7 | 0.82 | 6 | 1.2 | 0.6 | 6 |
| #7 | 3.2 | 1.2 | 6 | 2.6 | 0.80 | 6 | 0.8 | 0.8 | 6 |
| #8 | 0.8 | 0.3 | 6 | 0.3 | 0.41 | 6 | 0.7 | 0.4 | 6 |
| #9 | 0.8 | 0.4 | 6 | 0.4 | 0.58 | 6 | 0.6 | 0.2 | 6 |
| #10 | 3.3 | 0.4 | 6 | 3.5 | 1.05 | 6 | 2.7 | 0.9 | 6 |
| #11 | 1.2 | 0.4 | 6 | 2.1 | 0.86 | 6 | 2.3 | 0.8 | 6 |
| #12 | 3.7 | 0.6 | 6 | 2.7 | 1.21 | 6 | 1.5 | 0.9 | 6 |
| #13 | 2.3 | 0.6 | 6 | 2.3 | 1.25 | 6 | 1.3 | 0.7 | 6 |
| #14 | 1.7 | 0.6 | 6 | 1.7 | 0.98 | 6 | 1.5 | 0.9 | 6 |
| #15 | 2.5 | 1.9 | 6 | 3.1 | 0.38 | 6 | 2.9 | 1.5 | 6 |
| #16 | 3.4 | 0.4 | 6 | 3 | 0.45 | 6 | 2.0 | 1.3 | 6 |
| #17 | 3.2 | 0.8 | 6 | 2.5 | 0.89 | 6 | 2.3 | 1.4 | 6 |
| #18 | 3.7 | 0.4 | 6 | 2 | 0.74 | 6 | 2.8 | 1.4 | 6 |
| #19 | 1.7 | 0.4 | 6 | 1.8 | 0.93 | 6 | 2.2 | 1.0 | 6 |
| #20 | 1.6 | 0.8 | 6 | 2.2 | 0.88 | 6 | 2.0 | 1.0 | 6 |
| #21 | 3 | 0.7 | 6 | 2.6 | 0.77 | 6 | 3.0 | 0.3 | 6 |
| #22 | 3 | 0.6 | 6 | 3.3 | 0.41 | 6 | 3.0 | 0.3 | 6 |
| #23 | 1.3 | 0.3 | 6 | 1.9 | 0.66 | 6 | 2.8 | 0.5 | 6 |
| #24 | 3.7 | 0.3 | 6 | 3.3 | 0.69 | 6 | 3.3 | 0.4 | 6 |
| #25 | 3.9 | 0.9 | 6 | 3.3 | 0.41 | 6 | 3.8 | 0.6 | 6 |
| #26 | 0.9 | 0.5 | 6 | 1.4 | 0.92 | 6 | 2.2 | 0.9 | 6 |
| #27 | 1.6 | 0.6 | 6 | 1.9 | 0.58 | 6 | 2.4 | 0.7 | 6 |
| #28 | 1.2 | 0.3 | 6 | 1.7 | 0.75 | 6 | 1.4 | 0.7 | 6 |
| #29 | 0.8 | 0.4 | 6 | 1.4 | 0.86 | 6 | 1.3 | 0.8 | 6 |
| #31 | 0.9 | 0.2 | 6 | 0.4 | 0.20 | 6 | 0.8 | 0.5 | 6 |
| #36 | 0.4 | 0.2 | 6 | 0.6 | 0.38 | 6 | 1.9 | 1.0 | 6 |

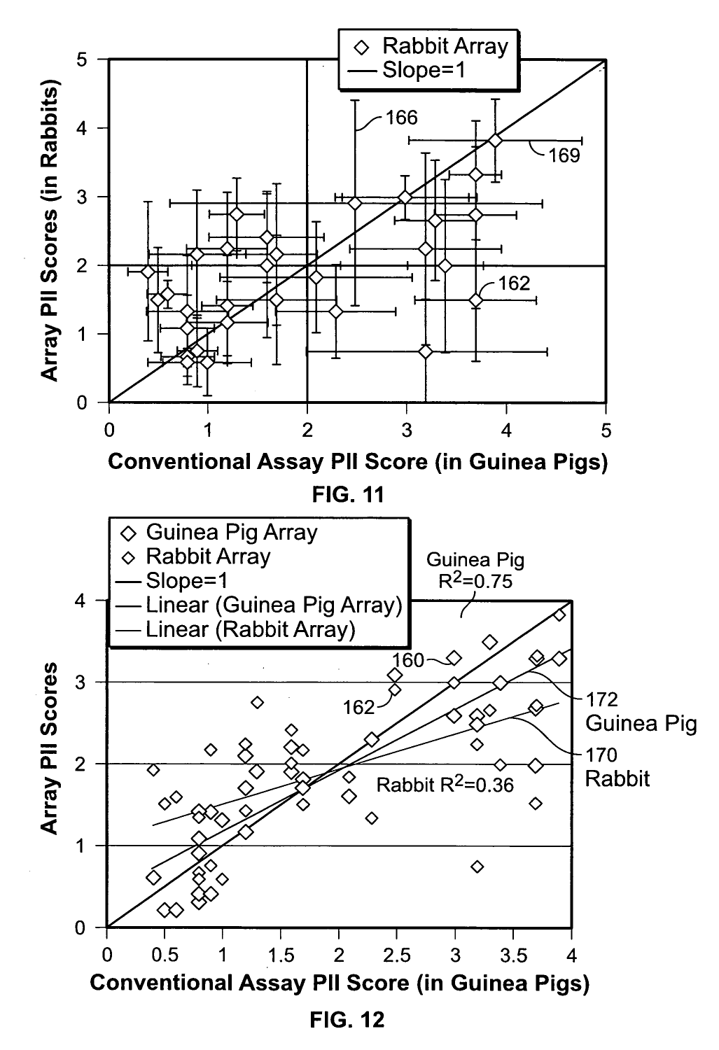

In FIG. 10 and FIG. 11, which display the above Table II data, the diagonal line is a reference line with slope of 1, shown as a reference for perfect correlation. The dots (160 for guinea pig, see FIG. 10; 162 for rabbit, see FIG. 11) are the averaged values over all the replicate animals of the same species for each test formulation. The vertical lines (164 for guinea pig, see FIG. 10; 166 for rabbit, see FIG. 11) with end bars and passing through the dots each indicate the standard deviation for PII scores for that test formulation for the array test animals. The horizontal lines (168 for guinea pig, see FIG. 10; 169 for rabbit, see FIG. 11) with end bars and passing through the dots each indicate the standard deviation for PII scores for that test formulation for the conventional test animals. FIG. 12 shows the combination of the rabbit data (smaller dots 162) and the guinea pig data (larger dots 160). A linear regression line 170 is shown for the rabbit data and a linear regression line 172 is shown for the guinea pig data. The correlation (R²=0.36 for rabbit data; R²=0.75 for guinea pig data) between the array data and the conventional test data was best when the array data were collected from the same species as the conventional test (e.g., comparing guinea pig data with guinea pig data) rather than from a different species, as would be expected.

Statistical analysis of the guinea pigs data showed that there was no significant difference in mean irritation scores between the two protocols. However, the variability from the arrays was slightly higher than that of the conventional protocol.

Because people skilled in the art often use mean irritation indices as categorical data, where formulations are deemed acceptable if their PII scores fall below a certain cutoff (typically 2-3), in this study data were also analyzed as categorical data and both protocols compared in terms of their ability to categorize formulation PII scores above or below a cutoff of 2. Contingency analysis was done to analyze the correspondence and agreement between the array test and convention test data. The correspondence analysis showed a high level of agreement between the two protocols (>90%). Thus, the array test is a viable alternative to the conventional test. With the array test, higher throughput protocol to measure PII is possible in animals such as hairless guinea pigs, as well as rabbits and other animals.

The entire disclosure of each patent, patent application, and publication cited in this document is hereby incorporated herein by reference. Embodiments of the present invention have been described with specificity. It is to be understood that various combinations and permutations of various parts and components of the schemes disclosed herein can be implemented by one skilled in the art without departing from the scope of the present invention. It is to be further understood that when an object or material is mentioned in an embodiment, a plurality or combination of the object or material is also contemplated as useful unless specified otherwise.

What is claimed is:

1. A method for irritation testing of formulations on skin, comprising: applying a patch with a flexible substrate having a group of formulations on skin of an animal such that the patch conforms to the skin for the formulations to contact the skin and the patch is affixed by an adhesive to the skin for a predetermined period of time.

2. The method of claim 1 wherein the formulations are arranged in groups in an array containing at least 9 formulations on the substrate.

3. The method of claim 2 wherein the formulations are each at least 12 mm² in area and spaced apart from a neighbor formulation with a gap of at least 2 mm.

4. The method of claim 2 wherein the patch has at least 15 formulations, the formulations are each 15 mm² to 200 mm² in area and spaced apart from a neighbor formulation with a gap of 2 mm to 5 mm.

5. The method of claim 2 comprising peeling off a protective cover covering the formulation to expose the formulations before applying the formulation on the skin.

6. The method of claim 2 wherein the formulations are attached to the substrate by an adhesive that is not part of the formulation and the method comprising pressing the patch on the body surface without exposing the body surface to any adhesive between the formulations.

7. The method of claim 2 wherein the formulations each are secured to the substrate by an individual adhesive no bigger in area than the formulation such that there is no adhesive not covered by the formulations between neighboring formulations.

8. The method of claim 2 wherein the formulations are secured to the substrate by spacer such that no adhesive contacts the skin between neighboring formulations upon application on skin.

9. The method of claim 2 further comprising pressing formulations that have a curved surface facing the skin before application thereon.

10. The method of claim 2 further comprising allowing the formulations to contact the skin for a total of 1 day to 7 days.

11. The method of claim 2 further comprising scoring for edema and erythema to evaluate irritation.

12. A method for making a patch for irritation testing of formulations on skin of an animal, comprising: securing a group of formulations on a flexible substrate such that the patch conforms to the skin for the formulations to contact the skin, and providing an adhesive for attaching the patch to the skin for a predetermined period of time.

13. The method of claim 12 comprising arranging the formulations into a regular array containing at least 9 formulations on the substrate.

14. The method of claim 12 comprising making the formulations each at least 12 mm² in area and spacing apart from a neighbor formulation with a gap of at least 2 mm.

15. The method of claim 12 wherein the patch has at least 15 formulations, the formulations are each 15 mm²to 200 mm² and spaced apart from a neighbor formulation with a gap of 2 mm to 5 mm.

16. The method of claim 12 comprising covering the formulations with a removable protective cover, which can be removed to expose the formulations before applying the formulation on the skin.

17. The method of claim 12 comprising attaching the formulations to the substrate by adhesive such that there is no adhesive exposed to the skin between the formulations.

18. The method of claim 12 comprising securing the formulations to the substrate through spacers.

19. The method of claim 12 further comprising providing the formulations thereof each a curved surface facing the skin before application thereon.

20. The method of claim 12 further comprising wherein the adhesive is capable of allowing the formulations to contact the skin for a total of 1 day to 7 days.

21. The method of claim 12 further comprising using a programmable robotic system to dispense formulations on the substrate.

22. A flexible patch for irritation testing of formulations on skin, comprising: a flexible substrate; a group of formulations secured on the flexible patch for application on skin of an animal such that the patch conforms to the skin for the formulations to contact the skin, the patch having an adhesive for adhering to the skin for a predetermined period of time.

23. The patch of claim 22 wherein the formulations are grouped into an array containing at least 9 formulations on the substrate.

24. The patch of claim 22 wherein the formulations are each at least 12 mm² in area and spaced at least 2 mm away from a neighbor formulation.

25. The patch of claim 22 wherein the patch has at least 15 formulations, the formulations are each 15 mm to 200 mm² in area and spaced 2 mm to 5 mm away from a neighbor formulation.

26. The patch of claim 22 further comprising a removable protective cover covering the formulation.

27. The patch of claim 22 wherein the formulations are attached to the substrate by adhesive.

28. The patch of claim 22 wherein the formulations each are secured to the substrate by an individual adhesive amount no bigger in area than the formulation such that there is no adhesive not covered by formulations between neighboring formulations.

29. The patch of claim 22 wherein the formulations are secured to the substrate through spacers such that no adhesive contacts the skin between neighboring formulations.

30. The patch of claim 22 wherein at least some of the formulations have a curved surface facing the skin before application thereon.

31. The patch of claim 22 wherein the adhesive is capable of allowing the formulations to contact the skin for a total of 1 day to 7 days.

32. The patch of claim 22 comprising adhesive on the substrate facing the skin wherein the adhesive encircles the group but no adhesive is between formulations on the substrate.
